(19) United States
(12) Patent Application Publication
(10) Pub. No.: US 2008/0228073 A1
(43) Pub. Date: Sep. 18, 2008

(54) SYSTEM AND METHOD FOR OPTOACOUSTIC IMAGING OF PERIPHERAL TISSUES

(76) Inventors: Ronald H. Silverman, West Nyack, NY (US); Jeffrey A. Ketterling, New York, NY (US)

Correspondence Address: KEITH D. NOWAK, CARTER LEDYARD & MILBURN LLP, 2 WALL STREET, NEW YORK, NY, 10005, US

(21) Appl. No.: 12/046,975

(22) Filed: Mar. 12, 2008

Related U.S. Application Data
(60) Provisional application No. 60/894,292, filed on Mar. 12, 2007.

(51) Int. Cl. A61B 8/00; A61B 6/00

(52) U.S. Cl. 600/437; 600/476

(57) ABSTRACT

The system and method for optoacoustic imaging of peripheral tissues provides an assembly for emitting radiation on a tissue and an OCT system coupled to said assembly. The OCT system images tissue concurrent with the emission of radiation on the tissue.

### PRIORITY AND RELATED APPLICATION

This application claims priority to U.S. Provisional Patent Application Ser. No. 60/894,292, filed Mar. 12, 2007, entitled “System and Method for Optoacoustic of Imaging of Peripheral Tissues,” which is hereby incorporated by reference in its entirety.

### FIELD OF THE INVENTION

The present invention relates to systems and methods for medical imaging. In particular, the present invention relates to a system and method of imaging biological materials including superficial tissues at high resolution and with multiple modalities.

### BACKGROUND OF THE INVENTION

Advancements in biomedical imaging have significantly improved the detection of potentially life threatening diseases. Biomedical imaging encompasses many different areas including x-ray computed tomography, magnetic resonance imaging (MRI), ultrasound, optical coherence tomography (OCT) and other techniques. Recently, OCT has gained recognition in biomedical imaging and has been used to image tissues such as the eye, skin, oral cavity, respiratory tract, gastrointestinal tract and bladders.

OCT has been developed for noninvasive cross-sectional imaging in biological systems. OCT emits light on tissue surfaces and captures or detects the reflected light to provide non-invasive, high-resolution sectional images in real time. Specifically, OCT is based on low-coherence interferometry to produce two dimensional images of optical scattering from internal tissue microstructures.

Known optoacoustic imaging systems use light pulses to produce acoustic sources in biological tissues with enhanced optical absorption. Diagnostic ultrasonic waves can propagate in biological tissues with minimal distortion, interrogate the optically-activated acoustic sources, and deliver diagnostic information to the surface of tissue, whereby the tissues with enhanced or differential optical absorption may be detected or imaged. It is the object of the present invention to improve known optoacoustic imaging techniques in order to provide enhanced detection ability and improved resolution of a detected image.

### BRIEF SUMMARY OF THE INVENTION

The present invention describes a system and method for optoacoustic imaging of peripheral tissues. The method includes combining an ultrasound transducer assembly with an OCT system, emitting at least one ultrasound radiation wave from the assembly and emitting light radiation from the OCT system simultaneous with the emitted ultrasound radiation wave, acquiring diagnostic data from the combined effects of the ultrasound radiation wave and the light radiation and imaging the data. The system comprises an assembly including a fiber optic light source integrated with an ultrasound transducer, and an OCT detection system coupled to said assembly. The OCT system images tissue concurrent with the emission of both ultrasound and light radiation on the subject tissue.

### BRIEF DESCRIPTION OF THE DRAWING

FIG. 1 is a schematic diagram illustrating a prior art optical coherence tomography system;

FIG. 2 shows an annular array pattern transducer of the prior art; and

FIG. 3 is shows a schematic diagram of an assembly of the present invention.

### DETAILED DESCRIPTION OF THE INVENTION

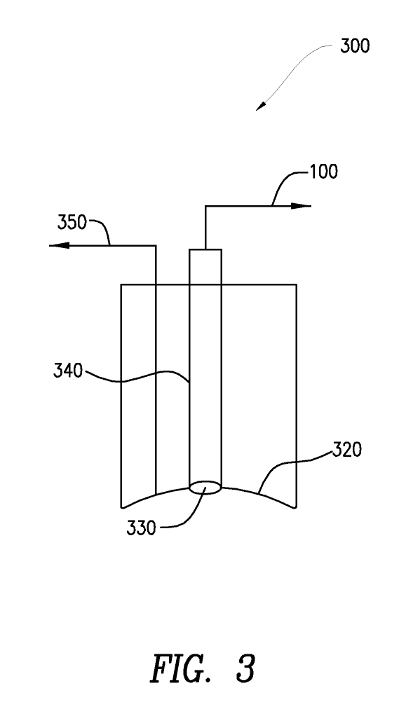

The present invention describes a system and method for optoacoustic imaging of peripheral tissues. The method of the present invention combines an OCT system with an ultrasound transducer to visualize propagation of the acoustic wave through tissue. The resulting image provides enhanced images compared to the prior art images. The present invention is realized by an optoacoustic device or assembly 300 shown in FIG. 3. The device 300 combines an ultrasound transducer, represented by piezoelectric element 320, with a coherent fiber optic bundle 340 and lens 330, see FIG. 3.

Ultrasound interacts well with light. An ultrasound wave is a pressure and density disturbance propagating through a tissue at the speed of sound. This exerts a spatially and temporally dependent tissue compression and rarefaction that causes corresponding oscillations in optical properties, including absorption, scattering and refractive index. A focused ultrasound beam may thus “tag” or encode light with which it interacts and the encoded signal can be differentiated from background using an optical receiver.

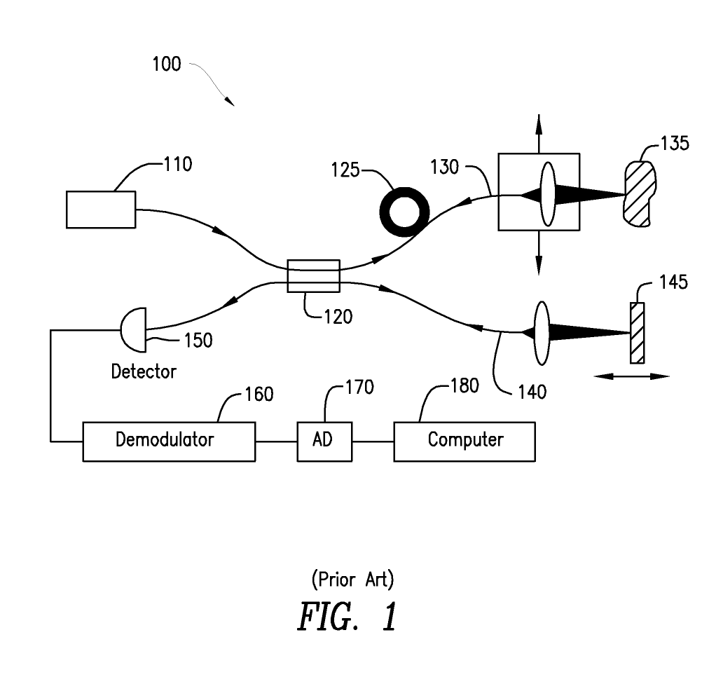

Referring now to FIG. 1, a traditional high-speed optical coherence tomography (OCT) scanner systems 100 of the prior art is shown. The OCT scanner 100 includes a low-coherence optical light source 110, an interferometer 120 including a beam splitter, and a detector 150. The scanner system 100 can further include a demodulator 160, a digitizer 170 and a computer 180. The scanner system 100 may further be a Fourier domain system ultilizing a spectometer or a swept-source laser. The optical source 110 may be a superluminescent diode (SLD), semiconductor sources such as LEDs or ELEDs or the like. SLDs generally have a short coherence length of about ˜10 μm. Preferable optical sources 110 include near infrared sources that tend to penetrate tissue deeper than visible wavelengths. The interferometer 120 shown here is a fiber optic Michelson interferometer using a 50/50 beam splitter; however, the interferometer 120 may be of any type known in the art.

The beam splitter of the interferometer 120 receives light from the optical source 110 and transmits or splits some fraction of light (here being 50%) into two arms, a sample interferometer arm 130 and a reference interferometer arm 140. Tissue sample 135 is placed at the end of a sample interferometer arm 130, while a reference 145 is placed at the end of a reference interferometer arm 140. Radiation reflected back from each of the sample 135 and the reference 145 are then recombined by the interferometer 120 and transmitted to the photo diode detector 150. The interferometric signal is modulated at a high frequency by Doppler shift and piezoelectric modulation. Specifically, a piezoelectric transducer (PZT) 125 in the sample arm 130 provides further piezoelectric modulation to the interferometric signal. Interferometric modulation of the output intensity is only detected by the detector 150 when the delays of both the sample interferometer arm 130 and the reference interferometer arm 140 are nearly matched. Output from the detector 150 is then frequency-filtered by the demodulator 160 to separate the desired interferometric signal from noise outside the signal bandwidth and produces an envelope of the interferometric signal. The signal is then digitized at Analog to Digital converter (AD) 170 by converting received analog signals into digital signals which are stored on a computer 180.

The OCT scanner 100 as described above is one basic example of a non-invasive, highly sensitive interferometric technique used to measure light reflected from tissues and produce cross-sectional images. Such imaging has proven useful in imaging a plethora of internal body tissues. However, to improve the OCT scanner system 100 of the prior art, the present invention adds to the OCT system 100 a device 300 combining an ultrasound transducer 320 with a fiber optic 340 and lens 330 to provide further enhancements in biomedical imaging.

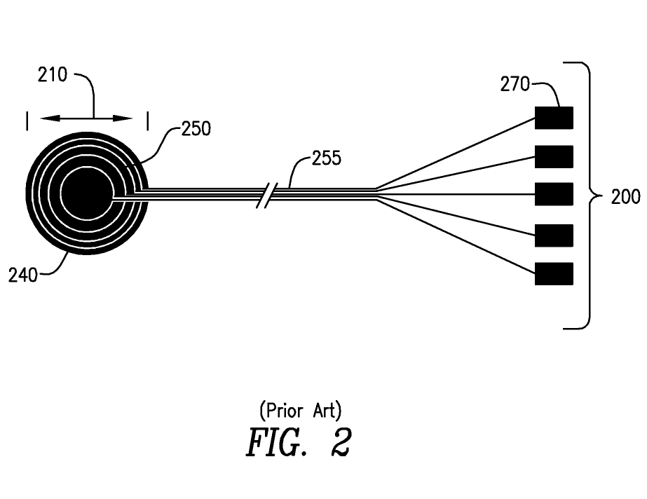

Suitable transducers for the present invention include focused single-element ultrasound transducers or an annular array ultrasound transducer known in the art. FIG. 2 shows the annular array ultrasound transducer 200 which is described in recently issued patent application to Ketterling entitled “System and Method for Design and Fabrication of a High Frequency Transducer,” Application No. 11/136,223. The annular array transducer 200 has annuli 240, apertures 210, annuli spacing 250, electrical traces 255 and contact pads 270. In some cases, a hole may be created in the center of the transducer 200; the center of the transducer 200 is located within annuli 240. Suitable transducers employed in the present invention will be high frequency broadband probes having a frequency between about 20 to about 50 MHZ. Such transducers will be suitable for high-resolution imaging and will be coupled to a pulser/receiver 350, see FIG. 3. The transducer 200 is combined with a fiber optic 340 and lens 330, see FIG. 3, for light transmission.

FIG. 3 shows an optoacoustic device or assembly 300. The device 300 includes fiber optic bundle 340, lens 330 and piezoelectric element 320 of the ultrasound transducer. The lens 330 serves to focus the light to a point coincident with the focus of the ultrasound transducer. In certain embodiments, the light is focused through the hole created in the center of the annuli of the annular transducer.

The device 300 integrates the ultrasound transducer with the fiber optic 340 and lens 330 by either making the foci coincide or by aligning axes. When the device 300 is made by making the foci coincide, the ultrasound transducer is integrated with the fiber optic 340 and lens 330 by mounting the fiber optic 340 and lens 330 external to the ultrasound transducer and orienting fiber optic 340 and lens 330 external to the ultrasound transducer so the foci of each coincide.

When the device 300 is made by aligning the optical axis and the ultrasound axis, the device 300 may be made in two ways. One way to manufacture the device 300 is to create a hole in the center of the transducer to accommodate the fiber optic 340 and lens 330. A second way is to align the fiber optic bundle 340 and lens 330 with an optically transparent central zone in the transducer. For example, the transducer may be composed of a thin piezoelectric film of PVDF 320. FIG. 3 shows the piezoelectric element 320 of the transducer connected to the pulser/receiver 350, while the fiber optic bundle 340 is coupled to the OCT scanner system 100.

In use, device 300 is acoustically coupled to tissue using a fluid medium that is fully or partially transparent at the optical wavelength of the light source. Suitable medium may include water, saline or oil.

The device 300 can be used to acquire data to visualize the effect of the propagation of acoustic wave(s) through tissue while the tissue is also being scanned by an OCT light source. This method uses high-speed OCT to detect ultrasound-induced refractive-index disturbances propagating at the speed of sound in tissue. Here, OCT images are acquired in two ways. One way is by having an OCT acquisition rate sufficient to obtain multiple images as a single ultrasound acoustic pulse traverses a tissue. The penetration depth of OCT is about 1 mm and the speed of sound is about 1540 m/s in biological tissues. Accordingly, the time for an acoustic pulse to traverse a 1 mm thick tissue is about 0.65 μsec. In order to visualize the pulse traversing the tissue, multiple OCT images must be taken in a preferred time period. For example, an exemplary minimum number of ten images may be taken for a sample interval of 65 nsec or a sample rate of 15 Mhz.

The second way to acquire the OCT images is by using a strobe technique. Here, a series of ultrasound pulses are emitted and one OCT image is acquired for each pulse with increasing time delays for successive pulses. Accordingly, each image captures the ultrasound-induced refractive index perturbation at different stages of its traversal of the tissue. To acquire an OCT image using the strobe technique the formula t=t₀+nt_{d} is used. Where n is the pulse number, t₀ is the time for the ultrasound pulse to reach the surface of a tissue after being emitted by the ultrasound transducer, and t_{d} is a sample interval short enough to capture images of ultrasound-induced refractive index disturbances as the pulse traverses the tissue. In an example, t_{d} would be defined as 65 nsec or less. Thus, by using an ultrasound pulse repetition frequency of 1 KHz, only 10 msec would be needed to acquire a series of images making tissue motion artifacts negligible.

The device of the present invention may also be used to acquire data to measure the viscoelastic properties of tissue. In this method, the ultrasound transducer portion, as represented by element 320, of the device 300 of the present invention is used to generate radiation force while tissue is simultaneously imaged with the OCT system 100. The use of ultrasound radiation force to generate tissue displacement is known and is described in a paper by Kathryn Nightingale et al. at J. Acoust. Soc. AM. 2001;110:625-634. The transducer is excited to a higher energy and for a longer duration when used to generate radiation force than when used to image. Here, since the OCT system 100 provides for high-resolution tissue imaging the spatial peak temporal average intensity may be significantly lower than 1000 W/cm² that was used in the Nightingale paper. Therefore, it is anticipated that the present invention would allow visualization of much smaller tissue displacements than shown in Nightingale.

The device of the present invention may further be used to acquire images tracking the acoustic propagation wave and viscoelasticity in acoustic radiation force imaging, as well as conventional pulse/echo ultrasound images and conventional OCT images. Accordingly, different tissue properties can be combined to provide fused images (image fusion) displaying these differing properties.

In an alternative embodiment, the device of the present invention may also be used to build an acoustic microscope. Here, ultrasound frequencies of 100 MHz or more with a short focal length and low f-ratio may be used to build the acoustic microscope suitable for imaging of tissue sections. The microscope would work on the principles describe above but would use a much higher ultrasound frequency, e.g. 500 MHz, that would provide sufficient resolution to image cellular details. At this frequency, penetration is limited and the technique would be used only for imaging thinly sliced (10-100 μm) tissue sections, cell cultures, etc. The ultrasound transducer would have a low f-ratio so as to provide good lateral resolution.

While the invention has been described by way of example and in terms of specific embodiments it is not so limited and is intended to cover various modifications as would be apparent to those skilled in this art area. Therefore, the scope of the appended claims should be accorded the broadest interpretation so as to encompass all such modifications.

1. A method for optoacoustic imaging of peripheral tissues comprising:
focusing a light source at points on the peripheral tissue;
focusing ultrasound radiation on the same point;
detecting reflections from the tissue in response to the focused light source; and
monitoring distortions in the detected reflections caused by the focused ultrasound radiation.

2. The method of claim 1, wherein the light source is emitted by an OCT system and the ultrasound radiation is emitted by an assembly, wherein the assembly is combined with the OCT system.

3. The method of claim 1, wherein the light source is emitted simultaneous to the emission of the ultrasound radiation.

4. The method of claim 1, wherein the ultrasound radiation is emitted by an assembly.

5. The method of claim 4, wherein the assembly comprises a fiber optic having a lens integrated with a transducer, said fiber optic having a lens having an axis and a focus, said transducer having an axis and a focus, said transducer having a center.

6. The method of claim 5, wherein the axis of the fiber optic having a lens is aligned with the axis of the transducer.

7. The method of claim 5, wherein the fiber optic having a lens is mounted external to the transducer.

8. The method of claim 7, wherein focus of the fiber optic having a lens coincides with the focus of the transducer.

9. The method of claim 7, wherein the transducer has a hole in the center of the transducer to accommodate the fiber optic having a lens.

10. The method of claim 7, wherein the center of the transducer is optically transparent.

11. The method of claim 10, wherein the center of the transducer is composed of a thin piezoelectric film.

12. A system for optoacoustic imaging of peripheral tissues comprising:
an assembly for emitting at least one ultrasound radiation wave on a tissue, said assembly further including a fiber optic having a lens integrated with a transducer; and
an OCT system, said OCT system coupled to said assembly, said OCT system providing at least one OCT image of said tissue, wherein said image is effected by the emission of ultrasound radiation on the tissue.

13. The system of claim 12, wherein multiple OCT images are obtained as a single ultrasound radiation wave is emitted from the assembly during a time period.

14. The system of claim 12, wherein more than one ultrasound radiation wave is emitted from the assembly during a time period.

15. The system of claim 12, wherein there are time delays for successive radiation waves emitted from the assembly.

16. The system of claim 12, wherein one OCT image is acquired for each radiation wave emitted from the assembly.

17. The system of claim 12, wherein one OCT image is acquired at t=t₀+nt_{d}.

18. A device comprising:
a fiber optic, said fiber optic having a lens, said fiber optic having an axis and a focus; and
an ultrasound transducer, said transducer having an axis and said focus, said transducer being integrated with said fiber optic forming an assembly, and detection means for detecting variations in reflections from tissue within said focus caused by interaction between said fiber optic radiation and said ultrasound radiation.

19. The device of claim 18, wherein the axis of the fiber optic is aligned with the axis of the transducer.

20. The device of claim 18, wherein the fiber optic is mounted external to the transducer so that the focus of the fiber optic coincides with the focus of the transducer.
